(19) United States
(12) Patent Application Publication
(10) Pub. No.: US 2008/0089943 A1
(43) Pub. Date: Apr. 17, 2008

(54) USE OF ADSORBENT CARBON MICROSPHERES TO TREAT GASTROESOPHOGEAL REFLUX DISEASE

(76) Inventor: Xavier Frapaise, Rancho Santa Fe, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/840,108

(22) Filed: Aug. 16, 2007

Related U.S. Application Data
(60) Provisional application No. 60/838,932, filed on Aug. 17, 2006.

(51) Int. Cl. A61K 9/14; A61K 31/435; A61P 1/04

(52) U.S. Cl. 424/489; 514/338

(57) ABSTRACT

Disclosed herein is the use of adsorbent carbon microspheres for the treatment of gastroesophogeal reflux disease. The adsorbent carbon microspheres may be administered alone or in combination with a proton pump inhibitor.

### RELATED APPLICATION

This application claims priority under 35 U.S.C. 119(e) to U.S. Provisional Application No. 60/838,932, filed Aug. 17, 2006, the entire contents of which are incorporated herein by reference.

### BACKGROUND

1. Field of the Invention

One aspect of the present invention relates to the treatment of gastroesophogeal reflux disease (GERD) and symptoms associated with GERD using adsorbent carbon microspheres.

2. Description of the Related Art

Gastroesophogeal reflux disease (GERD) is one of the most prevalent gastrointestinal disorders. Gastroesophageal reflux disease (GERD), a condition in which the liquid content of the stomach regurgitates into the esophagus, affects an estimated 5 to 7% of the global population including men, women and children (prevalence based occurrence of heartburn once per day). GERD may be caused by acid reflux and/or bile reflux. In some patients GERD damages the lining of the esophagus and if not properly managed, can evolve towards erosive esophagitis (EE), Barrett's esophagus and adenocarcinoma. GERD is an affliction that frequently affects patient quality of life.

Acid reflux is characterized by backflow of caustic stomach acids into the esophagus, which may be caused in particular by a decrease in the tone of the lower esophageal sphincter (LES). The refluxed liquid usually contains acid and pepsin produced by the stomach; it can also contain bile acids that have backed-up into the stomach from the duodenum (duodenogastric reflux). Acid reflux into the esophagus can cause esophagitis and other complications.

Bile reflux is characterized by backflow of bile into the stomach from the duodenum and potentially into the esophagus. Bile reflux may be caused by improper functioning of the pyloric valve, which may be due to damage caused by gastric surgery or obstruction by a peptic ulcer. Bile reflux can cause esophagitis as well as inflammation of the stomach. Bile reflux often accompanies acid reflux.

GERD is most often treated using proton pump inhibitors (PPIs) or H₂ antagonists. PPIs are highly effective in the treatment of GERD and are safe and well-tolerated even in elderly patients. However a significant GERD patient population (as much as 30%) is or has become resistant to treatment with PPIs and H₂ antagonists. Current treatment options include lifestyle changes, antacids, acid blockers, H₂ Receptor Antagonists (H₂RAs), proton pump inhibitors (PPIs), and endoscopic and surgical procedures.

### SUMMARY

One embodiment disclosed herein includes a method of treating one or more symptoms of gastroesophogeal reflux disease, comprising administering to a subject adsorbent carbon microspheres having a particle size of about 0.01 to about 2 mm.

Another embodiment disclosed herein includes a method of reducing bile reflux from a duodenum into a stomach, the method comprising introducing into the duodenum adsorbent carbon microspheres having a particle size of about 0.01 to about 2 mm.

Another embodiment disclosed herein includes a method of treating one or more symptoms of gastroesophageal reflux disease (GERD) in a patient who continues to be symptomatic after administration of a proton pump inhibitor (PPI), the method comprising administering to the patient adsorbent carbon microspheres having a particle size of about 0.01 to about 2 mm.

Another embodiment disclosed herein includes a method of treating one or more symptoms of gastroesophageal reflux disease (GERD), comprising co-administering to a subject a proton pump inhibitor (PPI) and adsorbent carbon microspheres having a particle size of about 0.01 to about 2 mm.

### DETAILED DESCRIPTION

### Definitions

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional such as a Medical Doctor (i.e. Doctor of Allopathic medicine or Doctor of Osteopathic medicine). or a Doctor of Veterinary Medicine to attempt to cure, or at least ameliorate the effects of, a particular disease or disorder or to prevent the disease or disorder from occurring in the first place.

As used herein, a “dosage” refers to an amount of therapeutic agent administered to a patient.

As used herein, a “daily dosage” refers to the total amount of therapeutic agent administered to a patient in a day.

As used herein, the term meq means milliequivalents(s).

As used herein, the term “therapeutic agent” means a substance that is effective in the treatment of a disease or condition. For example, the therapeutic agent(s) can be proton pump inhibitors (PPIs), H₂ antagonists, antacids, acid blockers and H₂ Receptor Antagonists (H₂RAs).

Treatment of GERD

In some embodiments, GERD or symptoms associated with GERD are treated or ameliorated by administering a therapeutic amount of adsorbent carbon microspheres to a patient. As used herein, an “adsorbent carbon microsphere” is a particle having a spherical or spheroid-like shape whose composition is mostly carbon and which has adsorbent properties. In some embodiments, the carbon microspheres have diameters from about 0.01 to about 2 mm. In some embodiments, the diameters are from about 0.02 to about 1 mm. In still other embodiments, the diameters are from about 0.05 to about 0.8 mm. In a typical embodiment, the carbon microspheres can have diameters from about 0.1 to about 0.5 mm. For example, the carbon microspheres can have diameters from about 0.2 to about 0.4 mm.

In some embodiments, the adsorbent carbon microspheres have a specific surface area of about 700 m²/g or more, such as determined by a BET (Brunauer-Emmett-Teller theory model) method (Brunauer et al. “Adsorption of Gases in Multimolecular Layers”, J. Am. Chem. Soc., 1938, 60(2), 309-319, which is incorporated herein by reference in its entirety). In some embodiments, the specific surface area is from about 700 m²/g to about 2500 m²/g. In a typical embodiment, the specific surface area is from about 1400 m²/g to about 1900 m²/g. For example, the specific surface area may be from about 1500 m²/g to about 1800 m²/g. In some embodiments, the volume of pores in the carbon microspheres having a pore diameter of about 20 to about 15,000 nm are from about 0.04 mL/g to about 0.10 mL/g.

In some embodiments, the total amount of acidic groups on the carbon are from about 0.30 to about 1.20 meq/g. In a typical embodiment, the total amount of acidic groups on the carbon is from about 0.30 to about 0.80 meq/g. For example, the total amount of acidic groups on the carbon may be from about 0.40 to about 0.70 meq/g. In some embodiments, the total amount of basic groups on the carbon are from about 0.20 to about 1.00 meq/g. In a typical embodiment, the total amount of acidic groups on the carbon can be from about 0.30 to about 0.80 meq/g. For example, the total amount of acidic groups on the carbon may be from about 0.35 to about 0.65 meq/g.

Suitable forms of adsorbent carbon microspheres are also described in U.S. Pat. Nos. 4,681,764 and 6,830,753 and U.S. Application Publication Nos. 2005/0112114; 2005/0079167; and 2005/0152890; all of which are incorporated herein by reference in their entirety.

In one embodiment, the adsorbent carbon microspheres are AST-120, available under the trade name KREMEZIN® from Kureha Corp. (Japan). AST-120 is a spherical activated carbon produced from pitch, such as by the process disclosed in U.S. Pat. No. 4,681,764. AST-120 has a particle size of about 0.2 to about 0.4 mm and is a homogeneous spherical particle (not a spherical particle produced by granulating a carbon powder).

Using a standardized adsorption assay, AST-120 demonstrated a high adsorption rate of bile salts and bile salts derivatives. Using a similar adsorption assay, AST-120 demonstrated greater than 90% adsorption ability for neuro and vasculo-modulators such as histamine and serotonin, as well as for gastrointestinal stimulants such as caffeine, tyramine, tryptamine and linoleic acid. AST-120 did not show a significant adsorption of lipopolysaccharides but did exhibit a high adsorption rate for N-formyl-met-leu-phe, which mediates neutrophil inflammatory response.

While not being bound by any particular theory, it is believed that adsorbent carbon microspheres as described above act to treat symptoms of GERD by adsorption of bile acids and small molecular weight toxins and mediators of inflammation from the gastrointestinal tract. It is also believed that adsorbent carbon microspheres serve to reduce bile content in the duodenum, thereby reducing the potential for bile reflux. In some embodiments, the adsorbent carbon microspheres are effective without causing certain side effects such as constipation or adsorption of beneficial intestinal enzymes (e.g., α-amylase).

In some embodiments, administration of adsorbent carbon microspheres to a patient suffering from GERD results in decrease of one or more GERD related symptoms, including but not limited to, daytime heartburn, nighttime heartburn, painful swallowing, difficulty swallowing, bad tasting fluid in mouth, and chest pain. In some embodiments, administration of adsorbent carbon microspheres to a patient suffering from GERD results in an improvement in the GERD Symptom Assessment Scale (GSAS). In some embodiments, administration of adsorbent carbon microspheres to a patient suffering from GERD results in an increase in esophageal pH. In some embodiments, administration of adsorbent carbon microspheres to a patient suffering from GERD results in a decrease in esophageal bilirubin.

In some embodiments, adsorbent carbon microspheres such as AST-120 are administered in combination with one or more therapeutic agent(s) to treat GERD. In some embodiments, adsorbent carbon microspheres such as AST-120 can be used to treat GERD in patients who continue to be symptomatic on a standard dose of proton pump inhibitor. For example the PPI can be selected from a non-limiting list consisting of Omeprazole, Lansoprazole, Esomeprazole, Pantoprazole, Rabeprazole and the like.

In some embodiments, adsorbent carbon microspheres such as AST-120 are administered to a patient taking from 10 mg to 40 mg of Omeprazole per day. In another embodiment, the adsorbent carbon microspheres are administered to a patient taking from 15 mg to 90 mg of Lansoprazole per day. In another embodiment, the adsorbent carbon microspheres are administered to a patient taking from 20 mg to 80 mg of Esomeprazole per day. In another embodiment, the adsorbent carbon microspheres are administered to a patient taking from 20 mg to 80 mg of Esomeprazole per day. In another embodiment, the adsorbent carbon microspheres are administered to a patient taking from 10 mg to 80 mg of Pantoprazole per day. In another embodiment, the adsorbent carbon microspheres are administered to a patient taking from 20 mg to 60 mg of Rabeprazole per day.

The oral dosage of the adsorbent carbon microspheres, either administered alone or in combination with another therapeutic agent, may be from about 1 to 20 grams per day. In some embodiments, the daily dosage of the adsorbent carbon microspheres may be divided into multiple administrations (e.g. into two to four portions daily). In some embodiments each portion can be from about 1 g to about 5 g (e.g., about 2 g to about 3 g). In a typical embodiment, a patient takes from about 2 g to about 3 g of adsorbent carbon microspheres (e.g., AST-120) three times daily. In some typical embodiments, the patient also takes 10 mg to 20 mg of Omeprazole, 15 mg to 60 mg of Lansoprazole, 20 mg to 40 mg of Esomeprazole, 20 mg to 40 mg of Pantoprazole, or 20 mg to 40 mg of Rabeprazole per day.

In some embodiments, the adsorbent carbon microspheres can be prescribed or administered at a specific dosage per day. In other embodiments, the patient can be instructed to take the composition when he or she experiences one or more symptoms related to a condition being treated. For example, the patient may be instructed to take a composition when he is experiencing severe pain.

As used herein, by administration in “combination,” it is meant that the adsorbent carbon microspheres are in the patient at the same time as one or more therapeutic agents may be found in the patient's bloodstream or stomach, regardless of when or how the adsorbent carbon microspheres and therapeutic agents are actually administered. In one embodiment, the adsorbent carbon microspheres and the therapeutic agent(s) are administered simultaneously. In one such embodiment, administration in combination is accomplished by combining the adsorbent carbon microspheres and the therapeutic agent(s) in a single dosage form. In another embodiment, the adsorbent carbon microspheres and the therapeutic agent(s) are administered sequentially. In one embodiment the adsorbent carbon microspheres and the therapeutic agent(s) are administered through the same route, such as orally. In another embodiment, the adsorbent carbon microspheres and the therapeutic agent(s) are administered through different routes, such as one being administered orally and another being administered i.v.

Production of Adsorbent Carbon Microspheres

Adsorbent carbon microspheres suitable for use as described herein may be produced by any suitable method, including but not limited to the following:

First, a bicyclic or tricyclic aromatic compound or a mixture thereof having a boiling point of 200° C. or more is added as an additive to a pitch such as a petroleum pitch or a coal pitch. The whole is heated and mixed, and then shaped to obtain a shaped pitch. Thereafter, the shaped pitch is dispersed and granulated in hot water at 70 to 180° C., with stirring, to obtain a microspherical shaped pitch. The aromatic additive is extracted and removed from the shaped pitch by a solvent having a low solubility to the pitch but a high solubility to the additive. The resulting porous pitch is oxidized by an oxidizing agent to obtain a porous pitch subject to heat infusibility. The resulting infusible porous pitch is treated at 800 to 1000° C. in a gas flow such as steam or carbon dioxide gas reactive with carbon to obtain a porous carbonaceous substance.

The resulting porous carbonaceous substance is then oxidized in a temperature range of 300 to 800° C., preferably 320 to 600° C., in an atmosphere containing 0.1 to 50% by volume, preferably 1 to 30% by volume, particularly preferably 3 to 20% by volume, of oxygen. The substance is thereafter reduced in a temperature range of 800 to 1200° C., preferably 800 to 1000° C., in an atmosphere of a non-oxidizable gas to obtain the final product.

In one alternative, the adsorbent carbon microspheres may be produced from a resin instead of a pitch. More details of suitable production processes and suitable products may be found in U.S. Pat. Nos. 4,681,764; 6,830,753; and U.S. Application Publication No. 2005/0112114, filed May 26, 2005, all of which are incorporated herein by reference in their entirety. Suitable adsorbent carbon microspheres are commercially available from Kureha Corp., and are sold in Japan under the trade name KREMEZIN® (also known as AST-120).

Pharmaceutical Compositions

For use as described herein, adsorbent carbon microspheres may be administered to the gut of a subject by any suitable means. In one embodiment, the adsorbent carbon microspheres are administered orally. Formulations for oral administration may include, but are not limited to, the free flowing microspheres, granules, tablets, sugar-coated tablets, capsules, suspensions, sticks, divided packages, or emulsions. In the case of capsules, gelatin capsules, or if necessary, enteric capsules may be used. In the case of tablets, the formulations may advantageously be adapted to break into the original fine particles inside the body. In the case of free flowing microspheres, the formulations may be in a sachet that is opened immediately prior to ingestion. The adsorbent may be used as a mixture with an electrolyte-controlling agent, such as an aluminum gel or KAYEXALATE® (Winthrop Lab, U.S.A.) or other agents. The microspheres may be ingested with the aid of a liquid or soft food (e.g., apple sauce).

Certain preparations for oral use can be obtained by mixing one or more excipients with adsorbent carbon microspheres as described herein and processing the mixture after adding suitable auxiliaries, if desired, to obtain tablets or dragee cores. The term “carrier” material or “excipient” herein can mean any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration.

Excipients can include, by way of illustration and not limitation, diluents, disintegrants, binding agents, adhesives, wetting agents, polymers, lubricants, glidants; substances added to mask or counteract a disagreeable texture, taste or odor; flavors, dyes, fragrances, and substances added to improve appearance of the composition. Acceptable excipients include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. Suitable excipients for injectable solutions include, without limitation, water, alcohols, polyols, glycerol, and vegetable oils. The pharmaceutical compositions can additionally include preservatives, solubilizers, stabilizers, wetting agents, emulsifiers, sweeteners, colorants, flavorings, buffers, coating agents, or antioxidants.

A variety of techniques for formulation and administration can be found in Remington: The Science and Practice of Pharmacy (20^{th} ed, Lippincott Williams & Wilkens Publishers (2003)), which is incorporated herein by reference in its entirety.

The dosage form used may be any amount of adsorbent carbon microspheres suitable to achieve the desired therapeutic result. In some embodiments, each unit dose is from about 1 g to about 5 g (e.g., about 2 g to about 3 g). In some embodiments, dosages of the adsorbent are individually packaged so as to preserve the absorptivity of the material. For example, divided packaging may be used such as described in more detail in U.S. Pat. No. 5,686,081, which is incorporated herein by reference in its entirety. The divided packaging may contain unit doses of the adsorbent carbon microspheres in their free flowing form.

### EXAMPLES

### Example 1

A human patient suffering from GERD is administered adsorbent carbon microspheres having particle sizes between about 0.05 and about 2 mm. The adsorbent carbon microspheres are administered orally as 2 g of free flowing microspheres three times daily. The symptoms of GERD are reduced after repeated administration of the spherical carbon.

### Example 2

A human patient is diagnosed as suffering from bile reflux from the duodenum into the stomach. The patient is administered adsorbent carbon microspheres having particle sizes between about 0.05 and about 2 mm. The adsorbent carbon microspheres are administered orally as 2 g of free flowing microspheres three times daily. The amount of bile refluxed into the stomach is reduced after repeated administration of the microspheres.

### Example 3

A double-blind, randomized, placebo-controlled crossover study is conducted to assess the efficacy of AST-120 in patients with gastroesophageal reflux disease (GERD) who continue to be symptomatic on a standard dose of a proton pump inhibitor (PPI).

The patients included in the study have a history of GERD symptoms resistant to a standard course of PPI, which is confirmed during the 2 week screening period. The patients have an abnormal esophageal bilirubin level but normal esophageal pH values.

AST-120 or placebo sachets 2 g three times daily (TID) are taken immediately before each of 3 daily meals for 4 complete weeks following a two-week screening period. Placebo is identical in taste and appearance to AST-120. After a 1 week washout period patients cross over to the opposite blinded treatment (2 g TID for 4 complete weeks). During the washout period all patients receive placebo in a blinded manner.

The double-blinding is achieved through the use of active study drug and placebo identical in taste and appearance. Secondary containers for the investigational product have coded labels based on the randomization scheme. Primary packaging for the investigational product does not identify whether the product is active or placebo.

Concomitant medications (if any) are administered at least 30 minutes before AST-120 or placebo administration. Patients continue to receive the previously prescribed PPI throughout the duration of the trial and are allowed up to 6 Gelusil tablets daily as rescue medication.

A hematology panel consisting of complete blood count (CBC) with differential, hematocrit (Hct), hemoglobin (Hgb), red blood count (RBC) indices (mean corpuscular volume (MCV), and platelet count, are performed at the Screen Visit only, to ensure that the patient's health status is appropriate for study entry. Thereafter, patients are evaluated throughout the study for clinical safety assessments including changes in physical exam, vital signs, GERD related symptoms and adverse events.

A patient is defined as having completed study treatment if he/she has received investigational product and is followed for safety through the last on-site visit of the crossover period (week 10).

The primary efficacy endpoint is a reduction in the severity of GERD symptoms in patients receiving AST-120 assessed by comparing the symptom scores on the validated GERD Symptom Assessment Scale (GSAS)

GSAS, reported by Rothman et al., “Symptoms associated with gastroesophageal reflux disease: development of a questionnaire for use in clinical trials”, Dig Dis Sci. 2001 July; 46(7):1540-1549 (incorporated herein by reference in its entirety), is the most comprehensive evaluative symptom scale developed so far GERD assessment. It is a 15-item GERD-specific scale that focuses not only on the predominant but also on associated symptoms (e.g. frequency of episodes, intensity of symptoms, level of distress), is self-assessed and has been developed with significant patient input to cover all relevant symptoms. GSAS is administered on every on-site visit. The scale has been developed carefully and is well validated. It has been shown to have acceptable reliability (intra-class correlation coefficient>0.70) and content and construct validity.

Secondary Efficacy Endpoints include the reduction in the severity of GERD symptoms in patients receiving AST-120 assessed by patient self assessment using a daily diary. Reduction of symptom including percent days without heartburn, percent daytime period without heartburn, percent nighttime period without heartburn, percent change in SF-36 score (a brief and comprehensive generic, quality of life questionnaire, or rating scale), and amount of rescue medication (Gelusil) taken per day.

A patient is defined as having completed study treatment if he/she has received investigational product and is followed for safety through the last on-site visit of the crossover period (week 10).

Study

Initially, 20 patients with PPI resistant GERD are randomized to take AST-120 or placebo. The age group of the patients is from 18 to 80 years of age and the body weight of the patients is from 40 to 100 kg (88 to 220 lbs).

Inclusion Criteria

Criteria for inclusion in the study are as follows: the patients have a recent history of GERD related symptoms (at least twice weekly) that are confirmed during prior screening; the patients have a recent history of 8 week proton pump inhibitor (PPI) treatment without significant symptom improvement (PPI treatment continues throughout the study); abnormal bilirubin level as assessed by Bilitec; normal esophageal pH value (pH less than 4.0 for less than 4.2% of the time calculated over a 24 hr period); platelet count (thrombocytes) greater than 100,000 μL; and normal hemoglobin (Hgb) and hematocrit (Hct) levels.

Exclusion Criteria

Criteria for exclusion in the study are as follows: concurrent GI or other pathology which could interfere with the course of the study (e.g., erosive esophagitis, malabsorption, cirrhosis, ascites, bleeding, ulcer, diabetes, scleroderma, non-GI myopathy or neuropathy etc.); patients with cancer or undergoing chemotherapy for the treatment of cancer; patients with a history of upper GI surgery; patients with GERD complications such as stricture of the esophagus; contraindication to continued PPI treatment; patients requiring the concomitant use of NSAIDs for the duration of the study; uncontrolled systemic disease; diagnosis of a psychiatric disorder within the past 2 years and not on a stable dose of medication for at least 6 months; other major physical or psychiatric illness in previous 6 months as determined by the treating physician; known hypersensitivity or contraindication to any component of the test product (study drugs) or diagnostics used; participation in another study within eight (8) weeks prior to randomization; and patients unable to attend all visits required by the protocol.

Allowed Medications

Only the following medications are allowed: PPIs (changing from one PPI to another during the study is prohibited) and up to 6 Gelusil tablets per day, if needed, as a “rescue medication”

Disallowed Medications

The following therapies are discontinued and are not be given during this trial: H₂ receptor antagonists (H₂RAs); Non-steroidal anti-inflammatory drugs (NSAIDs); Baclofen; and antacids (OTC or prescription), except Gelusil as described above.

Clinical Evaluation

The clinical evaluation, which is performed at every on-site visit, is conducted by the investigator or qualified medical personnel and consists of assessing vital signs (blood pressure, heart rate, respiration rate and temperature) and conducting a physical exam. Height and weight is recorded at the Screen Visit (Visit 0) only. A comprehensive physical exam is performed at the screening visit while brief physical exams, consisting of a review of organ systems, are performed at each subsequent visit. For each system, expected and unexpected abnormalities are assessed and documented. Abnormalities are documented and recorded as adverse events if the abnormality is not captured as part of the patient's medical history or baseline physical exam, or if the abnormality worsens in frequency or severity since baseline assessment.

Study Discontinuation

Treatment is discontinued if: surgical intervention is required by serious treatment-emergent adverse event(s) or the patient withdraws his/her consent. To minimize study discontinuation due to persistence of severe symptoms, patients are allowed up to 6 Gelusil tablets daily as “rescue medication”. Discontinued patients are evaluated in a termination visit and no further study treatment is given.

AST-120

The study drug, AST-120 is composed of black, odorless spherical particles approximately 0.2-0.4 mm in diameter. AST-120 is practically insoluble in water, ethanol, diethyl ether, hexane, acetone, chloroform, and benzene. The pH of a water extract of AST-120 is neutral (6.0-8.0). AST-120 is prepared by oxidative and reductive heating of petroleum-derived activated carbon.

Placebo

The placebo consists of microcrystalline cellulose spheres (Celphere® CP-305) coated with a combination of dyes (FD&C Blue #1, FD&C Red #40 and FD&C Yellow #6) to match the black color of AST-120.

Packaging and Labeling

Both AST-120 and placebo are packaged in 2 g quantities in PVC-coated aluminum foil sachets. The sachets are attached in strips of 3 sachets each. Each day's dose is 3 sachets or one strip. The sachets are provided in cardboard secondary containers packaged as kits containing a 4 week supply of product for each patient (Weeks 1-4 and 7-10). A kit containing a 1 week supply of placebo sachets are packaged for each patient to be used during the 1 week blinded washout period (Week 6). Each kit contains an overage (2 additional days supply) to allow for flexibility in scheduling follow-up visits.

The kits are labeled in accordance with all applicable laws and regulations and are sequentially numbered (blinded/coded) according to the randomization schedule provided to the distributor. The labels will include a duplicate attached by perforation that is detached and affixed to the appropriate source document when the investigational product is dispensed to the patient.

Investigational Product Storage and Expiration

Stability data support a shelf-life of 24 months for AST-120 clinical trial materials (sachets). Stability of placebo gelatin capsules has been demonstrated out to 2 years at 25° C./60% relative humidity, and to 6 months (accelerated) at 40° C./75% relative humidity in sachets. It is anticipated that placebo is stable at room temperature for at least 2 years when stored in sachets.

Product Administration

Investigational product is shipped to the clinical sites in sufficient quantity to enroll 20 patients. The investigator distributes the kits in sequential order as patients are enrolled. The investigator detaches the duplicate label from the kit and affixes it to the appropriate source document. As patients complete the first full course of randomized treatment (4 weeks), the investigator distributes the kits for the 1 week washout period. At the end of the washout period the investigator distributes the crossover kits. The kits for the three treatment periods (i.e. initial randomized treatment, washout treatment and crossover treatment) is distinguished on the kit label using the designations A, B or C along with the sequential kit number. For example the first patient enrolled might be assigned kits 001A, 001B and 001C to be distributed at randomization, washout and crossover respectively, the second patient enrolled might be assigned kits 002A, 002B and 002C, etc.

Patients are instructed to take 3 sachets per day, each administration is immediately before a meal. For each administration, the patient tears open the sachet (along the pre-perforated “scissor-line” demarcation), drop the contents of the sachet directly on their tongue and swallow the investigational product using as much water as necessary to comfortably “wash down” the product and clear their mouths of any residue.

Statistical Methods

General Considerations

Data listings are created to support each table and to present all data as collected from the case report forms. Data from this study is analyzed using descriptive statistics for continuous variables and includes means, medians, standard deviations, minimums and maximums. Categorical variables are presented as frequency counts, and percents. Subgroup analyses is performed as appropriate.

Patient Characteristics

A detailed description of patient demographics and baseline characteristics is summarized using descriptive statistics. The summary table includes all patients who receive at least one dose of investigational product.

Efficacy Analysis

The efficacy analysis is done on those patients deemed evaluable per protocol (i.e., met all inclusion/exclusion criteria, randomized and treated correctly, adhered to study procedures, and received at least one dose of investigational product from both the initial and crossover arm).

The primary efficacy endpoint is the reduction in the severity of GERD symptoms in patients receiving AST-120 compared to placebo as assessed by the validated GSAS. The paired t-test and descriptive statistics are utilized to summarize the primary endpoint by treatment group.

The secondary efficacy endpoints, also utilize the appropriate descriptive statistical methodology to summarize each of these endpoints by treatment group. Descriptive statistics for continuous variables include the mean, standard deviation, median, minimum, and maximum; categorical variables are presented as frequency counts and percentages.

Safety Analysis

All patients receiving investigational product are included in the safety analysis. Laboratory values, physical examinations and vital signs are summarized over time and treatment group.

The primary safety endpoint is adverse events (AEs) deemed possibly, probably or definitely related to treatment with investigational product, compared between treatment groups. All AEs are presented in incidence tables by Medical Dictionary for Regulatory Activities (MedDRA) type and body system for the AST-120 and placebo groups. Events classified as possibly related, probably related, or definitely related to study drug administration are presented separately and combined with all other experiences. Frequency and percent of patients with serious adverse experiences are displayed.

Laboratory values, vital signs, AEs, prior and concomitant medications, and medical history data are provided in patient listings by treatment group.

The proportions of adverse events of any specific type or body system are compared using frequency counts and percentages.

In absolute terms, the frequency with which given laboratory values are outside the normal range and deemed clinically significant by the investigator, are computed and compared between treatment arms with frequency counts and percentages as part of the analysis of adverse events.

Pre-Study On-Site Visit

The patients go through a screening process two weeks prior to the start of the study. The patients sign an informed consent form and disclosure authorization form for protected health information.

The pre-study screening process includes: obtaining the patients medical history; collecting demographic information; a review of previous and concomitant medications, height and body weight measurement; hematology measurement; and a physical exam including the taking of vital signs is performed. Additionally, for pre-menopausal females a urine pregnancy test is required. All test results and assessments required to establish eligibility of the patient are obtained prior to enrollment and prior to dispensation of investigational product at the Baseline (Week 1) Visit. The patient diary is distributed at the Screen Visit and filled out for at least one week prior to the Baseline Visit to confirm occurrence of GERD related symptoms of at least twice a week.

Study On-Site Visit

The patients return at Week 1 (Visit 1) to establish the baseline and undergo randomization. This visit includes: confirmation of inclusion/exclusion criteria; review medical history; review of previous/concomitant medications; review of the inclusion/exclusion criteria; a physical exam including the taking of vital signs is performed; esophageal pH measurement is taken; review of patient diary filled out since the screening to confirm GERD symptom frequency; GSAS and SF-36, Bilirubin measurement (BILITEC); and upper endoscopy. Additionally, for pre-menopausal females a urine pregnancy test is conducted.

Once the patients fulfill the requirements of the inclusion/exclusion criteria check list they are randomized to take either study drug or placebo. The patients are then given a GERD symptom checklist, the study drug or placebo (blinded) and a patient diary.

The next on-site visit occurs at Week 5 (Visit 2) which is the end of the 1st treatment sequence. This visit includes: a review of previous/concomitant medications; a physical exam including the taking of vital signs; an esophageal pH measurement; a review of patient diary filled out since visit 1; GSAS and SF-36, and Bilirubin measurement (BILITEC). Additionally, for pre-menopausal females a urine pregnancy test is required.

The patients are then given the placebo (blinded) and a new patient diary. Additionally, an assessment of adverse events is undertaken.

The next on-site visit occurs at Week 6 (Visit 3) which is the end of the 1 week washout period. This visit includes: a review of previous/concomitant medications; GSAS and SF-36, and a physical exam including the taking of vital signs is performed. The patients who received the study drug (weeks 1-4) are given placebo (blinded) and the patient who received placebo (weeks 1-4) are given study drug (blinded) and a new patient diary. Additionally, an assessment of adverse events is undertaken.

The next visit on-site occurs at Week 10 (Visit 4) which is the end of the of crossover sequence. This visit includes: a review of previous/concomitant medications; a physical exam including the taking of vital signs is performed; an esophageal pH measurement; a review of patient diary filled out since visit 3; GSAS and SF-36, and Bilirubin measurement (BILITEC). Additionally, for pre-menopausal females a urine pregnancy test is again required. Finally, the patients undergo an assessment of adverse advents.

Early Termination

Early Termination visits are used when a patient withdraws from the study prior to Week 10 (Visit 4).

This visit includes: a review of previous/concomitant medications; a physical exam including the taking of vital signs; an esophageal pH measurement; a review of patient diary filled out since previous visit; and Bilirubin measurement (BILITEC). Additionally, for pre-menopausal females a urine pregnancy test is again required. Finally, the patients undergo an assessment of adverse advents.

Patient Symptom Record

The symptoms assessed on a daily basis include daytime heartburn, nighttime heartburn, painful swallowing, difficulty swallowing, bad tasting fluid in mouth, and chest pain. Each symptom present is rated as mild, moderate, severe, or disabling with the following definitions:

Mild Symptom easily tolerated and did not last long

Moderate Symptom caused some discomfort but did not interfere with usual activities

Severe Symptom caused much discomfort and interfered with usual activities

Disabling Symptoms unbearable and interfered considerably with usual activities

Detection of Bilirubin through Spectrophotometric Device (BILITEC)

The Bilitec 2000 (Synectics) detects the presence of bilirubin. This system consists of a fiber optic probe, 140 cm long and 3 mm in diameter. At the distal end it has a 9.5-mm-long, 5-mm-in-diameter head with a 2-mm orifice in which there are two diodes that emit light at different wavelengths: 470 nm (bilirubin value) and 565 nm (reference value). The probe is placed beside the esophageal pH electrode, 5 cm above the lower esophageal sphincter, and simple chest radiography is performed to check that the probes are placed correctly. The study is performed as an outpatient routine over 24 hours. Measurements are taken every eight seconds, and the mean between two measurements is calculated to obtain a total of 5,400 counts in 24 hours.

A measurement is considered positive (presence of bilirubin in the esophagus) when absorption is 0.14 units or greater. With values less than this, the measurement may be affected by the presence of mucosity or gastric particles, thus giving false-positive results.

The patient is instructed to eat three meals per day and to avoid foods and medication that might alter the study as a result of their colorimetric or pH characteristics. Alcohol consumption and smoking are also prohibited. The patient is given a diary in which to note the times of meals, situations of lying down and standing, and the appearance of symptoms. The probes are removed after 24 hours, and a software package (Esophogram; Gastrosoft, Irving, Tex.) is used to extract data and perform calculations to obtain numeric and graphic results.

Measurement of Esophageal pH

After an overnight fast, a pH probe with lower esophageal sphincter identifier is inserted via the nose and into the stomach. The lower esophageal sphincter identification manometry assembly is a simple system for water-perfused manometry using the combined pH and water-perfused pressure catheter. The pressure lumen is located 5 cm above the distal pH sensor. By using the station pull-through technique (0.5 cm increments), identification of the proximal margin of the lower esophageal sphincter is achieved. The pH sensor is placed 5 cm above the upper margin of the lower esophageal sphincter. The pH probe is connected to a digital portable recorder. A reference electrode is attached to the upper chest. Patients are instructed to keep a diary, recording meal times, position changes and the time and type of their symptoms while being encouraged to pursue their every day activities and usual diet. At the beginning and end of the study, the electrode and system are calibrated in standard solutions of pH 1 and 7. Reflux is defined as pH<4 and reflux time as the internal until pH is again greater than 4. The test is considered when the total percent time pH<4 and reflux time as the interval until pH will gain be greater than 4. The test is considered positive when the total percent time pH<4 is greater than 4.2%. The supine and erect time is considered abnormal if the duration ph<4 is greater than 1.2% or 6%, respectively.

Symptom index is calculated as the percentage of heartburn symptoms that correlate with acid reflux events. Symptom sensitivity index is calculated as the percentage of acid reflux events that are associated with GERD symptoms. Analysis of the recorded data is performed using standard commercially available computer software.

Results

The data demonstrate a reduction in the severity of GERD symptoms in patients receiving AST-120 assessed by comparing the symptom scores on the validated GERD Symptom Assessment Scale (GSAS) events patients treated with AST-120 compared with placebo. Additionally, the data demonstrate a reduction in percent days with heartburn, percent daytime period with heartburn, and percent nighttime period with heartburn for patients treated with AST-120 compared with placebo.

Although the invention has been described with reference to embodiments and examples, it should be understood that numerous and various modifications can be made without departing from the spirit of the invention. Accordingly, the invention is limited only by the following claims.

What is claimed is:

1. A method of treating one or more symptoms of gastroesophogeal reflux disease, comprising administering to a subject adsorbent carbon microspheres having a particle size of about 0.01 to about 2 mm.

2. The method of claim 1, further comprising identifying the subject as suffering from gastroesophogeal reflux disease.

3. The method of claim 1, further comprising identifying the subject as suffering from bile reflux.

4. The method of claim 1, wherein the adsorbent carbon microspheres are administered orally in a free flowing form.

5. The method of claim 1, wherein the symptoms are selected from one or more of daytime heartburn, nighttime heartburn, painful swallowing, difficulty swallowing, bad tasting fluid in mouth, and chest pain.

6. The method of claim 1, wherein the administration of the adsorbent carbon microspheres is sufficient to result in an improvement in the GERD Symptom Assessment Scale (GSAS).

7. The method of claim 1, wherein the administration of the adsorbent carbon microspheres is sufficient to result in an increase in esophageal pH.

8. The method of claim 1, wherein the administration of the adsorbent carbon microspheres is sufficient to result in a decrease in esophageal bilirubin.

9. The method of claim 1, wherein from about 1 gram to about 20 grams of the adsorbent carbon microspheres is administered daily.

10. The method of claim 1, wherein the adsorbent carbon microspheres are administered three times daily.

11. The method of claim 1, wherein each dose of adsorbent carbon microspheres administered is from about 2 grams to about 3 grams.

12. The method of claim 1, wherein the adsorbent carbon microspheres have a particle size of about 0.02 to about 1 mm.

13. The method of claim 1, wherein the adsorbent carbon microspheres have a particle size of about 0.05 to about 0.8 mm.

14. The method of claim 1, wherein the adsorbent carbon microspheres have a specific surface area of about 700 m²/g or more as determined by a BET (Brunauer-Emmett-Teller) method.

15. The method of claim 1, wherein the adsorbent carbon microspheres have a specific surface area of about 700 m²/g to about 2500 m²/g as determined by a BET (Brunauer-Emmett-Teller) method.

16. The method of claim 1, wherein the volume of pores in the adsorbent carbon microspheres having a pore diameter of about 20 to about 15,000 nm is about 0.04 mL/g to about 0.10 mL/g.

17. The method of claim 1, wherein the total amount of acidic groups in the adsorbent carbon microspheres is from about 0.30 to about 1.20 meq/g.

18. The method of claim 1, wherein the total amount of basic groups in the adsorbent carbon microspheres is from about 0.20 to about 1.00 meq/g.

19. A method of reducing bile content in a duodenum, the method comprising introducing into the duodenum adsorbent carbon microspheres having a particle size of about 0.01 to about 2 mm.

20. The method of claim 19, wherein the duodenum are in a human suffering from bile reflux.

21. The method of claim 20, further comprising identifying the human as suffering from bile reflux.

22. The method of claim 19, wherein introducing the adsorbent carbon microspheres into the duodenum comprises administering the adsorbent carbon microspheres to the human.

23. The method of claim 22, wherein the adsorbent carbon microspheres are administered orally in a free flowing form.

24. A method of treating one or more symptoms of gastroesophageal reflux disease (GERD) in a patient who continues to be symptomatic after administration of a proton pump inhibitor (PPI), the method comprising administering to the patient adsorbent carbon microspheres having a particle size of about 0.01 to about 2 mm.

25. The method of claim 24, wherein the PPI is selected form the group consisting of Omeprazole, Lansoprazole, Esomeprazole, Pantoprazole, and Rabeprazole.

26. The method of claim 24, wherein the adsorbent carbon microspheres are administered in one to four portions daily.

27. A method of treating one or more symptoms of gastroesophageal reflux disease (GERD), comprising co-administering to a subject a proton pump inhibitor (PPI) and adsorbent carbon microspheres having a particle size of about 0.01 to about 2 mm.

28. The method of claim 27, wherein the PPI is selected form the group consisting of Omeprazole, Lansoprazole, Esomeprazole, Pantoprazole, and Rabeprazole.

29. The method of claim 28, wherein the adsorbent carbon microspheres are administered in one to four portions daily.
